(19) United States
(12) Patent Application Publication
(10) Pub. No.: US 2015/0080816 A1
(43) Pub. Date: Mar. 19, 2015

(54) CLEANSING KIT FOR COLOSTOMY BAGS

(71) Applicant: HERMAN D TYREE, FLINT, MI (US)

(72) Inventor: HERMAN D TYREE, FLINT, MI (US)

(21) Appl. No.: 14/484,609

(22) Filed: Sep. 12, 2014

Related U.S. Application Data
(60) Provisional application No. 61/878,087, filed on Sep. 16, 2013.

(51) Int. Cl. A61F 5/442; A61F 5/445

(52) U.S. Cl. CPC A61F 5/442; A61F 5/445
USPC 604/334

(57) ABSTRACT

An apparatus for cleaning ostomy bags that includes a fluid supply, a selectively operable spray nozzle assembly, a conduit interconnecting the fluid supply and the spray nozzle assembly, a means for disconnecting the spray nozzle assembly from the conduit, and a means for disconnecting the conduit from the fluid supply. The spray nozzle assembly may include a body connectable to the conduit, an actuating handle for controlling the release of fluid, and a U-shaped tube connectable to the body for dispersing fluid. The U-shaped tube is selectively disconnectable from the body. The apparatus may include a carrying case a carrying case for storing and transporting the spray nozzle assembly, the conduit, and ostomy supplies. The fluid supply for the apparatus may be a refillable reservoir. The apparatus may include an electrically operated pump to provide the conduit with a supply of pressurized fluid.

### CROSS-REFERENCE TO RELATED APPLICATIONS

This application claims benefit of U.S. Provisional Application Ser. No. 61/878,087, filed on Sep. 16, 2013, which is incorporated herein in its entirety by reference.

### TECHNICAL FIELD

The disclosure relates to cleaning kits for ostomy bags, and more particularly, to cleaning kits for ostomy bags that are portable.

### BACKGROUND

Individuals that have undergone an ostomy or colostomy are repeatedly faced with the unpleasant task of emptying and cleaning ostomy bags that have become filled with bodily waste. This can occur multiple times a day. Such bags may be single use, disposable products or reusable. One difficulty associated with single use ostomy bags is the requirement for removal of used bags and reapplication of new bags at the ostomy site. To maintain an adequate seal between the entry orifice of such bags and the abdomen, the ostomy site must be thoroughly cleaned and an adhesive/sealant applied to the union between the abdomen and the ostomy bag. Repeated procedures of this type are time-consuming and frequently result in chronic irritation to the skin. Because of this, many ostomy patients prefer to utilize ostomy bags that may be opened, emptied, cleansed, and reclosed without needing to remove the ostomy bag from the ostomy patient's body.

Because of the configuration of ostomy bags, emptying and cleansing typically occurs by opening the bottom of the bag, and allowing the contents to drain into a suitable bathroom fixture, such as a commode. Using a nearby water supply and nozzle apparatus, water or other cleansing fluids may be directed into the interior of the bag until it is clean. Once cleaned, the bottom of the bag can be reclosed.

In addition to not having to remove the ostomy bag from the ostomy patient's body, there are important economic realities associated with reusing ostomy bags. For example, an ostomy patient utilizing single use ostomy bags may need to replace the ostomy bag several times in a day, at significant expense to the ostomy patient and his or her insurance provider. Cost-savings associated with the reuse of ostomy bags are, accordingly, substantial.

Devices for cleaning ostomy bags are well known. However, the devices are designed to be semi-permanently installed at water sources, such as a fresh water source available in a residential or institutional bathroom. Often, ostomy patient's need to empty and clean reusable ostomy bags while traveling, or are in a remote location where there is no access to running water.

### SUMMARY

Portable, self-contained apparatuses for cleaning ostomy bags are disclosed herein. An apparatus for cleaning ostomy bags includes a fluid supply, a selectively operable spray nozzle assembly, a conduit interconnecting the fluid supply and the spray nozzle assembly, a means for disconnecting the spray nozzle assembly from the conduit, and a means for disconnecting the conduit from the fluid supply. The spray nozzle assembly may include a body connectable to the conduit, an actuating handle for controlling the release of fluid, and a U-shaped tube connectable to the body for dispersing fluid. The U-shaped tube is selectively disconnectable from the body.

The apparatus may include a carrying case a carrying case for storing and transporting the spray nozzle assembly, the conduit, and ostomy supplies. The fluid supply for the apparatus may be a reservoir. The reservoir may be refillable. The apparatus may also include a corresponding electrically operated pump to provide the conduit with a supply of pressurized fluid. The electrically operated pump may be powered by a portable energy source. The portable energy source may be a battery, and the battery may be rechargeable.

### BRIEF DESCRIPTION OF THE DRAWINGS

The various features, advantages, and other uses of the present apparatus will become more apparent by referring to the following detailed description and drawings, in which:

FIG. 1 is a plain front view of an apparatus for cleaning ostomy bags;

FIG. 2 is a plain front view of the apparatus of FIG. 1 interfaced with a shower head and hung on nearby support;

FIG. 3 is a plain front view of the apparatus of FIG. 1 provided with a carrying case;

FIG. 4 is a plain front view of the apparatus of FIG. 1 provided with a portable fluid supply; and

FIG. 5 is a plain side view of the apparatus of FIG. 1 provided with the portable fluid supply.

### DETAILED DESCRIPTION

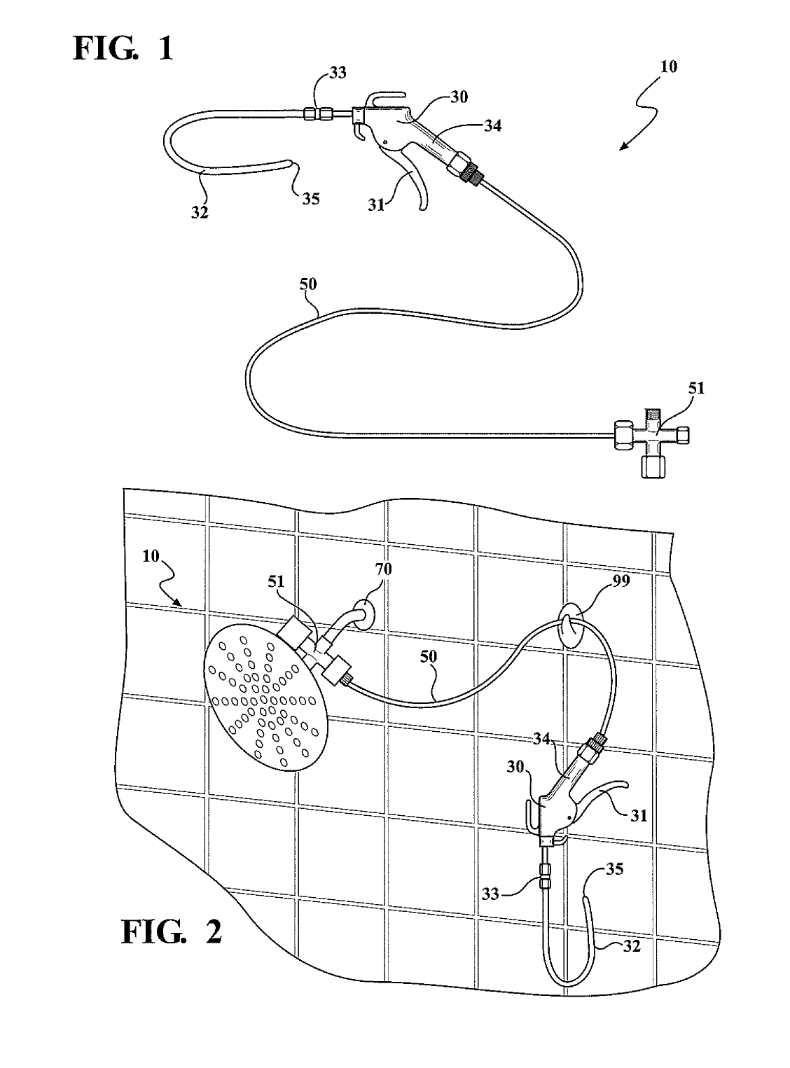

This description is directed to a self-contained and portable apparatus 10 for cleaning ostomy bags. As shown in FIGS. 1-5, the apparatus 10 can be removably attached to a plumbed water supply, such as a water faucet or a self-contained reservoir. The apparatus 10 may include both manual and powered pumping systems for providing a controlled, adequate source of water or cleaning fluid. This allows the apparatus 10 to be utilized in environments where there is no ready source of running water or electrical power.

The apparatus 10 is comprised of a spray nozzle assembly 30 that is connected to a fluid supply 70 through a supply conduit 50. The apparatus 10 can utilize a fluid supply 70, such as a household water supply, as the source of water for operation of the apparatus 10. The fluid supply may be associated with a typical bathroom shower, bathtub or commode, and the supply conduit 50 may be connected to the fluid supply 70 using a permanent, semi-permanent or temporary plumbing fitting. For example, the supply conduit 50 may be provided with a union 51 that interfaces with a shower head, sink faucet, shower head supply, sink supply, or commode supply. The union 51 may have a spring-loaded valve that shuts off the fluid supply when the supply conduit 50 is removed from the quick disconnect. In this fashion, the supply conduit 50 may be attached to or disconnected from the fluid supply without the requirement for tools, and with little or no spillage of water. Alternatively, the connection between the apparatus 10 and the fluid supply 70 may be in the form of a permanently plumbed valve having an outlet and a manually operated control, so that the fluid supply 70 to the supply conduit 50 may be controlled. For convenience, a hanger or support 98 may be affixed in proximity to the fluid supply 70 to allow hanging of the spray nozzle assembly 30 and supply conduit 50 near the fluid supply 70.

The spray nozzle assembly 30 may be equipped with an actuating handle or trigger 31. An outlet 32 of the spray nozzle assembly 30 is in the form of a U-shaped tube, which may be coated with a resilient material such as rubber or plastic. The outlet 32 may have a disconnect feature 33 that allows the outlet 32 to be disconnected from the spray nozzle assembly 30 for transportation or sanitization. A body 34 of the spray nozzle assembly 30 is fed by the supply conduit 50, which is attached to the body 34 of the spray nozzle assembly 30 using a permanent or removable plumbing fixture. A distal end 35 of the spray nozzle assembly 30 is formed with a flattened or reduced dimension outlet, which serves to disperse the flow of water exiting from the spray nozzle assembly 30 when the trigger 31 of the spray nozzle assembly 30 is operated.

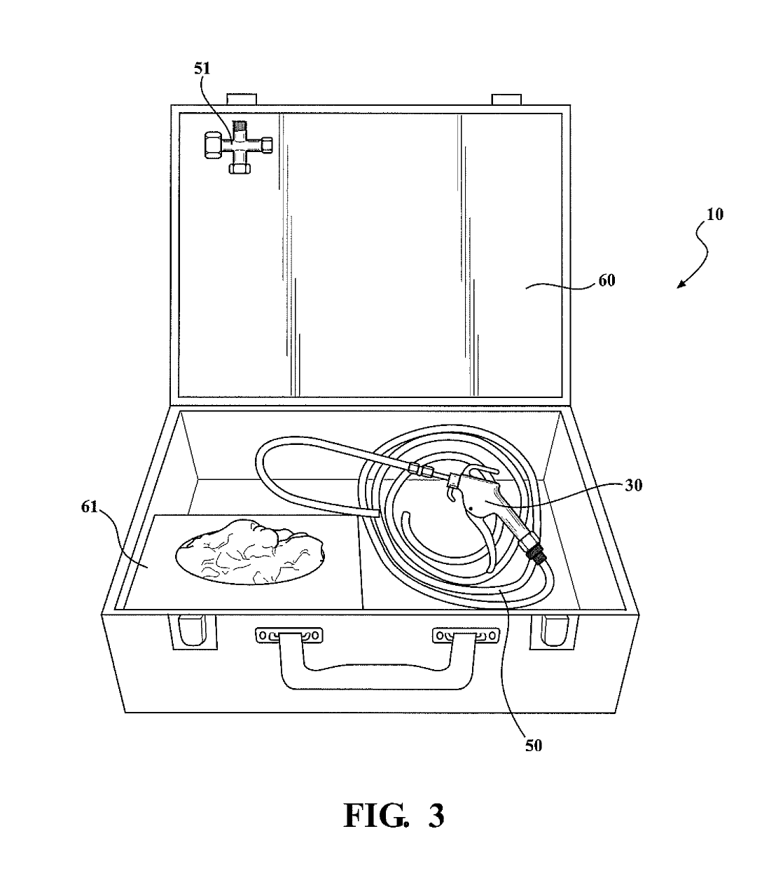

In use, the fluid supply 70 feeds water under normal household water supply pressures to the spray nozzle assembly 30, and the flow of water from the outlet 32 of the spray nozzle assembly 30 is controlled by the trigger 31 on the body 34 of the spray nozzle assembly 30. When desired, the supply conduit 50 may be disconnected from the fluid supply 70, and the supply conduit 50 and spray nozzle assembly 30 may be transported to another location for use. To facilitate transport of the apparatus 10, a carrying case 60, shown in FIG. 3, may be provided, which conveniently holds the spray nozzle assembly 30 and supply conduit 50 together with the patient's ostomy supplies 61 to any location providing a water source or supply. The interior of the carrying case 60 may be provided with one or more attach points or compartments, wherein the various components of the apparatus 10 may be conveniently stored.

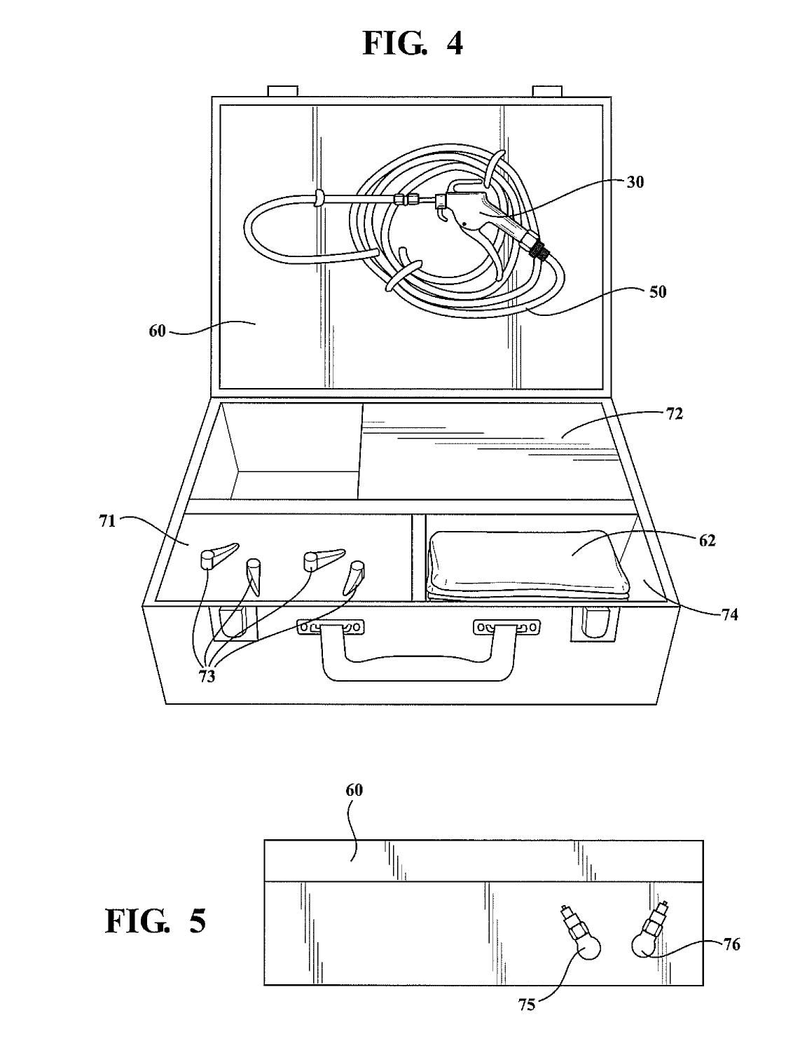

To allow for the apparatus 10 to be used in environments where there is no ready source of running water, the fluid supply 70 may be portable, as shown in FIGS. 4-5. When the fluid supply 70 is portable, the carrying case 60 is provided with a pump 71, a reservoir 72, a series of valves 73, and a storage compartment 74. The storage compartment 74 accommodates the patient's ostomy supplies 61, which may include replacement ostomy bags, rubber gloves, mastic, and cleaning pads that are usable at the ostomy site. The patient's ostomy supplies may fit within a second, smaller carrying case 62 that fits within the storage compartment 74, such as a small suitcase or briefcase.

The reservoir 72 may be refillable with water and may be provided with one or more resealable openings through which water, disinfectants, deodorants or other fluids may be directly added to the reservoir 72. The storage capacity of the reservoir 72 may vary but is generally in the range of three-quarters to one gallon of fluid. The reservoir 72 interconnects with an inlet quick disconnect 75 and an outlet quick disconnect 76. The inlet quick disconnect 75 may be connected by a suitable removable conduit, such as hosing or tubing, to an external water supply. The outlet quick disconnect 76 allows the reservoir 72 to communicate with the spray nozzle assembly 30. To facilitate discharge of fluid from the reservoir 72, the pump 71 is provided may be powered by a portable energy source, such as a battery, or by connection to conventional power outlets found in commercial or residential buildings. Electrical connectors may be provided on the exterior of the carrying case 60 to facilitate connection of external power to operate the pump 71 or to recharge the battery.

To use, the ostomy patient connects the spray nozzle assembly 30 to one end of the supply conduit 50 and the other end of the supply conduit 50 to the fluid supply 70. The ostomy patient then positions the ostomy bag in relation to a commode or other suitable drain before the ostomy patent opens the bottom of the ostomy bag, which should be resealable to allow for the contents of the ostomy bag to be emptied into the commode or drain. Next, the distal end 35 of the spray nozzle assembly 30 is inserted in the ostomy bag, and the ostomy patient uses the spray nozzle assembly 30 to spray the remaining contents of the ostomy bag with fluid from the fluid supply 99 to flush the remaining contents of the bag and clean the interior of the ostomy bag. As soon as the ostomy bag has been emptied and the cleaning operation is completed, the bottom of the ostomy bag is then resealed, allowing for continued use of the ostomy bad without the need for application of a new ostomy bag.

To use the apparatus 10 when the fluid supply 70 is portable, the ostomy patient similarly connects the spray nozzle assembly 30 to one end of the supply conduit 50. The other end of the supply conduit 50 is then connected to the outlet quick disconnect 76 of the reservoir 72. One or more valves are opened to allow the fluid in the reservoir 72 to communicate with supply conduit 50. The electric pump 71 is then turned on, providing pressurized fluid from the reservoir 72 to the supply conduit 50 and spray nozzle assembly 30, allowing the ostomy patient to clean the ostomy bag as previously described.

While this disclosure describes what is presently considered to be the most practical embodiments, it is to be understood that the apparatus 10 is not limited to the disclosed implements but, on the contrary, is intended to cover various modifications and equivalent arrangements included within the spirit and scope of the appended claims. The scope of the appended claims is to be accorded the broadest interpretation so as to encompass all such modifications and equivalent structures as permitted under the law.

What is claimed is:

1. An apparatus for cleaning ostomy bags comprising:
a fluid supply;
a selectively operable spray nozzle assembly; and
a conduit interconnecting the fluid supply and the spray nozzle assembly, wherein spray nozzle assembly and the fluid supply are disconnectable from the conduit.

2. The apparatus of claim 1, wherein the spray nozzle assembly further comprises:
a body connectable to the conduit and having a passageway for fluid from the fluid supply to pass through;
an actuating handle for controlling the release of fluid; and
a U-shaped tube connectable to the body for dispersing the fluid from the body, wherein the U-shaped tube is selectively disconnectable from the body.

3. The apparatus of claim 2, further comprising:
a carrying case for storing and transporting the spray nozzle assembly and the conduit.

4. The apparatus of claim 2, further comprising:
a carrying case for storing and transporting the spray nozzle assembly, the conduit, and ostomy supplies.

5. The apparatus of claim 1, wherein the fluid supply is a reservoir.

6. The apparatus of claim 5, wherein the reservoir is refillable.

7. The apparatus of claim 6, the reservoir further comprises:
a corresponding electrically operated pump whereby the electrically operated pump may be provide the conduit with a supply of pressurized fluid.

8. The apparatus of claim 7, wherein the electrically operated pump is powered by a portable energy source.

9. The apparatus of claim 8, wherein the portable energy source is a battery.

10. The apparatus of claim 9, wherein the battery is rechargeable.

11. The apparatus of claim 10, further comprising:
a carrying case for storing and transporting the spray nozzle assembly, the conduit, the reservoir, and the electrically operated pump.

12. The apparatus of claim 10, further comprising:
a carrying case for storing and transporting the spray nozzle assembly, the conduit, the reservoir, the electrically operated pump, and ostomy supplies.

13. An apparatus for cleaning ostomy bags comprising:
a refillable reservoir;
a selectively operable spray nozzle assembly;
a conduit interconnecting the refillable reservoir and the spray nozzle assembly, wherein spray nozzle assembly and the fluid supply are disconnectable from the conduit;
an electrically operated pump to provide the conduit with a supply of pressurized fluid; and
a carrying case for storing and transporting the spray nozzle assembly, the conduit, the refillable reservoir, the electrically operated pump, and ostomy supplies.

14. The apparatus of claim 13, wherein the electrically operated pump is powered by a portable energy source.

15. The apparatus of claim 14, wherein the portable energy source is a rechargeable battery.

16. The apparatus of claim 13, wherein the spray nozzle assembly further comprises:
a body connectable to the conduit and having a passageway for fluid from the fluid supply to pass through;
an actuating handle for controlling the release of fluid; and
a U-shaped tube connectable to the body for dispersing the fluid from the body, wherein the U-shaped tube is selectively disconnectable from the body.

17. The apparatus of claim 16, wherein the refillable reservoir and the electrically operated pump are permanently affixed within the carrying case.

18. The apparatus of claim 17, wherein the electrically operated pump is powered by a portable energy source.

19. The apparatus of claim 18, wherein the portable energy source is a rechargeable battery.

20. The apparatus of claim 19, wherein the ostomy supplies are housed within a second, smaller carrying case.
